(19) United States
(12) Patent Application Publication
(10) Pub. No.: US 2012/0035469 A1
(43) Pub. Date: Feb. 9, 2012

(54) SYSTEMS AND METHODS FOR THE MEASUREMENT OF SURFACES

(76) Inventors: Thomas J. Whelan, Longmont, CO (US); Stephen H. Sprigle, Marietta, GA (US); Robin D. Knight, Loveland, CO (US); Daniel H. Packard, Greeley, CO (US); Collin J. Horvat, Fort Collins, CO (US)

(21) Appl. No.: 13/226,724

(22) Filed: Sep. 7, 2011

Related U.S. Application Data
(63) Continuation-in-part of application No. 12/443,158, filed on Nov. 24, 2009.
(60) Provisional application No. 60/847,532, filed on Sep. 27, 2006.

(51) Int. Cl. A61B 6/00

(52) U.S. Cl. 600/425

(57) ABSTRACT

A portable, hand-held, non-contact surface measuring system comprises an image capturing element, at least four projectable reference elements positioned parallel to one another at known locations around the image capturing element, a processing unit, and a user interface. The invention further discloses a method for the noncontact surface measurement comprising projecting at least four references onto a target surface, capturing an image of the targeted surface and the projected references with the image transferring device, transferring the image to a processing unit, processing the image using triangulation-based computer vision techniques to correct for skew and to obtain surface measurement data, transferring the data to the user interface, modifying the data with the user interface. The systems and methods for the measurement of surfaces can be applied to the measurement of biological surfaces, such as skin, wounds, lesions, and ulcers.

### CROSS-REFERENCE TO RELATED APPLICATIONS

This application is a continuation-in-part of U.S. Patent Application Ser. No. 12/443,158, filed on Nov. 24, 2009, which published as U.S. Patent Publication No. 2010/0091104, on Apr. 15, 2010, and which is the National Phase of International Application No. PCT/US07/21032, filed on Sep. 27, 2007, which published in English as WO/2008/039539, on Apr. 3, 2008, and which claims benefit to U.S. Provisional Application No. 60/847,532 filed on Sep. 27, 2006. The disclosures of these documents are hereby incorporated by reference in their entirety as if fully set forth below.

### BACKGROUND OF THE INVENTION

The present invention refers generally to the characterization of surfaces, and more particularly to the systems and methods for the non-contact measurement of biological surfaces.

Chronic wounds, such as pressure ulcers and diabetic ulcers constitute a problem that affects approximately 20 percent of the hospitalized population in the United States. Chronic wounds limit the autonomy and quality of life experienced by the geriatric population, individuals with peripheral vascular disease, diabetes, or cardiac disease, individuals with spinal cord injuries, individuals with birth defects such as spina bifida, cerebral palsy, or muscular dystrophy, and post-polio patients. It is estimated that 25 percent of individuals with spinal cord injuries and 15 percent of individuals with diabetes will suffer from a chronic wound at some point in their lives. In addition to the cost in human suffering, there is a tremendous monetary cost also associated with the treatment of wounds and pressure ulcers. An estimated $20 billion is spent each year in the care of chronic wounds.

Improving the treatment strategy of chronic wounds by providing quantitative measurements for chronic wounds would greatly reduce cost and significantly improve the quality of life for those people who suffer from them. Specifically, proper and regular measuring of the size of a wound is crucial in determining the effects of ongoing treatment. Wound size information can lead to effective adjustments of treatment or reformulation of treatment to allow for optimal recovery. In addition, regular and accurate wound measurement would also provide practitioners a mechanism to maintain complete records of patient progress for the purposes of legal liability. Further, assessing whether a wound is healing, worsening, or remaining constant is often difficult because no rapid, noninvasive, and reliable method for measuring wounds currently exists. The lack of reliability in the measurement of wounds is largely attributable to the fact that defining a wound's boundary is often difficult endeavor, which depends highly on the subjective judgment of the human observer who performs the measurements. If a precise quantitative wound measurement system were available, caregivers would be able to speed wound healing by adjusting treatment modalities as the wound responds or fails to respond to treatment.

A great deal of research has been performed on the etiology and treatment of chronic wounds; however, treatment of chronic wounds is limited in part by the lack of a precise, noninvasive, and convenient means for the quantitative measurements for assessing wound healing. Examination of the current methods and devices for wound measurement demonstrate that the present technology can be divided into two classes. At one end of the spectrum, low technology methods for the measurement of chronic wounds, such as ruler-based methods and tracing-based methods, are easy to use; such methods, however, lack accuracy and involve contact with the wound. At the other end of the spectrum are high technology methods for chronic wound measurement, such as structured light technology and stereophotogrammetry, which both provide accurate and repeatable measurements but are expensive to implement and require extensive training to operate.

The most widely used wound assessment tools are plastic templates that are placed over the surface of the wound bed to permit the clinician to estimate the planar size of the wound. These templates range from a simple plastic ruler that provides a measurement of the major and minor axes of the wound to more sophisticated devices such as the Kundin gauge, which provides an estimate of the surface area and volume of the wound based on assumptions about the geometry of a typical wound. Of the template-based methods, ruler-based measurements are the most widely adopted method. When using a ruler, simple measurements are made and the wound is modeled as a regular shape. For example, the maximum diameter can be taken to model the wound as a circle. Measurements in two perpendicular directions can be taken to model the wound as a rectangle.

The Kundin gauge is another ruler-based device, which uses three disposable paper rulers set at orthogonal angles to measure length, breadth, and depth of the wound. The wound is modeled as an ellipse and the area is calculated as A=length* breadth*0.785. However, in real world situations, wounds are rarely regular enough to be modeled by one of these simple shapes. In addition, the repeatability in taking measurements largely depends on the chosen axes of measurement by the individual performing the measurements.

Another low cost method of wound measurement is the transparency tracing method. In this method two sterile transparent sheets are layered on top of the wound. The wound is outlined on the top sheet and the lower sheet is discarded. The area is approximated by laying the sheet over a grid and counting the number of squares on the grid covered by the outline of the wound. The area could also be estimated by using a planimeter or by cutting out and weighing the tracing. This method has more precision in terms of repeatability for both inter-rater and intra-rater tests, compared to ruler based methods. However, it is more time consuming. Additionally, the extended contact with the wound raises concerns about wound contamination, pain, and discomfort to the patient. Also, drawing on the wound surface can become difficult because of transparency clouding due to wound exudate. Other potential issues include difficulty and variations in identifying the wound edge, inaccurately tracing a wound due to a skin fold, or distorting the transparency sheet when conforming it to the wound surface.

Other methods are available that measure wound volume. A technique that has been used clinically to assess wound volume involves filling the wound cavity with a substance such as alginate. An alginate mold is made of the wound, and the volume of the wound can be calculated by either directly measuring the volume of the alginate cast by the use of a fluid displacement technique or the cast can be weighed and that weight divided by the density of the casting materials. A variation of this technique for measuring wound volumes involves using saline. A quantity of saline is injected into the wound, and the volume of fluid needed to fill the wound is recorded as the volume of the wound.

Although wound measurement methods employing a ruler, Kundin gauge, transparency tracing, alginate mold, or saline injection may be cost-effective and easy to perform, these contact methods of measuring a wound all share several significant problems. First, there is potential for disrupting the injured tissue when contact is made. Second, there is a significant risk of contamination of the wound site with foreign material or pathogenic organisms. In addition, fluids displaced through these contact methods could serve as a vector for the transmission of pathogens from the wound site to other patients or to the clinical staff. These contact-based measurements also fail to take into account additional characteristics of the wound beyond size, such as surface area, color, and the presence of granulation tissue.

Considering the limitations of contact-based measurement techniques, non-contact methods based on photographic methods of wound measurement have been explored. These methods are advantageous because they do not require contact with the wound. Therefore, the potential for damaging the wound bed or contaminating the wound site or its surroundings is eliminated. Currently, the available systems for making non-contact photographic measurements of wounds are expensive, utilize equipment that is cumbersome in a clinical setting (i.e., lacks mobility), require significant training for the operator, and entail meticulous set-up and calibration by the operator to obtain precise reproducible measurements.

The simplest photographic techniques are Polaroid prints. Color photographs of wounds have been further studied to determine the most effective type of film and lighting that can be used to document accurately the size of the wound and the status of the tissue in and around the wound. Tissue color and texture appear to provide clinicians with useful information about the health of the wound. In addition, two-dimensional image processing is useful for assessing wound parameters, such as surface area, boundary contours, and color. Photographs, however, in and of themselves fail to provide accurate calculations of the wound size or surface area

Current vision-based or photographic techniques make use of either stereophotogrammetry or the use of structured light. In stereophotogrammetry, two photographs of the same wound are taken from different angles. Using these images taken from known positions relative to the wound, a three dimensional (3-D) model of the wound can be reconstructed using a computer. The wound boundary is then traced, on the computer, and the software determines the area and volume of the wound. This field has melded the desirable characteristics of photography, such as the capability to represent object color and texture, with computers creating accurate 3-D representations of objects and surfaces. However, the stereophotogrammetry systems that have been previously described share the problems associated with non-contact photographic measurements of wounds, namely expense, cumbersome equipment, and significant preparation time to set-up and calibrate the equipment to create photographic data.

Structured light, on the other hand, consists of a specific pattern of light, such as dots, stripes or fringes. In the structured light technique, a specific pattern of light is projected onto a wound from a light source whose position is known relative to the light sensing equipment (i.e., a camera). The wound, which is illuminated with structured light, is photographed from a known angle. Using the image of the wound, the area and volume of the wound can be calculated based on the relative position of the wound within the structured light. Specifically, the topography of a surface can be determined through active triangulation repeated at many points on the surface. Each illuminated point can be considered the intersection point of two lines. The first line is formed by the ray of illumination from the light source to the surface. The second line is formed by the reflected ray from the surface through the focal point of the imaging device to a point on the image plane. Given the position and orientation of the light source and camera are known, the point on the surface can be computed through triangulation. The entire surface can be mapped by interpolating between multiple points on the surface. Multiple points are generated either by the algorithm sequentially computing the location of a single point that is scanned across the surface in multiple images or projecting a grid of points and processing the surface in a single image.

The requirements for accurate calculations using structured light technology include a known position and orientation of the illumination source, identifiable illumination points on the surface of interest, and a known position of the camera or other sensor so that the direction to the illuminated part of the surface. Given these requirements, structured light wound measurement systems share the same problems associated with stereophotogrammetry systems, including expense, cumbersome equipment, and significant preparation time to set-up and calibrate the equipment to create photographic data.

In addition, a substantial limitation of both the contact and non-contact methods for wound measurement currently available is that the practitioner is required to manually delineate the boundaries of the wound and the boundaries of different tissue types within the wound. Therefore, the present methods of wound measurement are highly subjective and depend largely upon the individual judgment of the practitioner assessing the wound. Reduction of human involvement in wound assessment is necessary because determination of wound parameters, such as wound surface area, should be automated in order to obtain a more objective and reproducible measure of the wound.

Considering the gap in technology that exists between the cost-effective contact-based wound measurement methods and the cumbersome, cost-prohibitive, non-contact-based methods of wound measurement employing structured light technology or stereophotogrammetry, there is a need for a portable, low-cost device that can reproducibly measure the two-dimensional characteristics of wounds. This need for point-of care technology for wound monitoring is further accentuated by the growing emphasis on treating persons with chronic wounds in skilled nursing facilities or in home-care environments. Further, development of a low-cost, portable, quantitative, non-contact method for reproducible wound measurement would prove useful for the documentation of the efficacy of a treatment strategy. Such documentation can limit liability of the care provider and allow timely changes in treatment strategy easier to justify in the managed-care environment.

According to some embodiments of the invention, the system can comprise a portable, self-contained, hand-held, low-cost, non-contact system for the reproducible measurement of surfaces.

### SUMMARY OF THE INVENTION

This invention relates to systems and methods for the measurement of surfaces. More particularly, the present invention discloses a self-contained, portable, hand-held, non-contact surface measuring system comprising an image capturing element, at least four projectable reference elements positioned parallel to one another at known locations around the image capturing element, a processing unit, and a user interface. The present invention further discloses a method for the non-contact surface measurement comprising projecting at least four reference points onto a target surface, locating the target surface and the projected references within the viewfinder of a image capturing device, capturing an image of the targeted surface and the projected references with the image transferring device, transferring the image to a processing unit, processing the image using triangulation-based computer vision techniques to correct for skew and to obtain surface measurement data, transferring the data to the user interface, and modifying the data with the user interface. The systems and methods for the measurement of surfaces can be applied to the measurement of biological surfaces, such as skin, wounds, lesions, and ulcers.

The present invention includes a portable, hand-held, non-contact self-contained surface measuring system capable of providing quantitative measurements of a target object on a target surface. The system comprises an image capturing element for capturing an image of at least a portion of the target object; at least four projectable reference elements for defining at least one characteristic of at least a portion the target object; a processing unit; and a user interface for displaying the captured image. Preferably, the target object is a wound, and the target surface is a biological element or surface. The characteristic can be the shape, size, boundary, edge(s), or depth of the target object, while the image capturing element can be a digital camera, personal digital assistant, or a phone.

Further, the present invention includes a method for providing quantitative measurements of a target object on a target surface. The method comprises providing a target object on a target surface; projecting at least four reference elements at least a portion of the target object; capturing an image of at least a portion of the target object; and defining at least one characteristic of at least a portion of the target object. The method can further comprise displaying the captured image on a user interface.

These and other features and advantages of the present invention will become more apparent upon reading the following specification in conjunction with the accompanying drawings.

### BRIEF DESCRIPTION OF THE DRAWINGS

The systems and methods and systems designed to carry out the invention will hereinafter be described, together with other features thereof.

The invention will be more readily understood from a reading of the following specification and by reference to the accompanying drawings forming a part thereof:

FIG. 1 illustrates a schematic of a non-contact system for the measurement of surfaces;

FIG. 2 illustrates an embodiment of a system for wound measurement;

FIG. 3 illustrates an embodiment of the image capturing device, within the system shown in FIG. 2;

FIG. 4A illustrates a screen capture of a detected wound boundary by the system shown in FIG. 2;

FIG. 4B illustrates user modification of wound boundary by dragging a control point;

FIG. 4C illustrates user modification of wound boundary by nudging a control point;

FIG. 5 illustrates a schematic of the boundary detection algorithm;

FIG. 6 illustrates coordinate detection geometry of laser points;

FIG. 7 illustrates the skew geometry of laser points;

FIG. 8A illustrates an original skewed image;

FIG. 8B illustrates an unskewed image;

FIG. 9A illustrates the conversion of a captured image to a grayscale image;

FIG. 9B illustrates an edge map of the captured image;

FIG. 9C illustrates a filled image of the captured image after 2 iterations;

FIG. 9D illustrates an edge map of the captured image after 3 iterations;

FIG. 9E illustrates a segmented image of the captured image after four iterations;

FIG. 9F illustrates a segmented boundary superimposed on the original image.

FIG. 10A illustrates Image 1, which was utilized in the repeatability tests;

FIG. 10B illustrates Image 2, which was utilized in the repeatability tests;

FIG. 11 illustrates the wound area measurements in the presence and absence of skew correction;

FIG. 12 illustrates an alternative embodiment that uses a divergent configuration of laser elements;

FIG. 13 illustrates the profile of laser elements with respect to the field of view of an image capturing device;

FIG. 14 illustrates coordinate detection geometry of laser points;

FIG. 15 illustrates the projection of laser elements in an image plane for a set of four calibration images; and

FIG. 16 illustrates a schematic of a device calibration algorithm.

### DETAILED DESCRIPTION OF THE PREFERRED EMBODIMENT

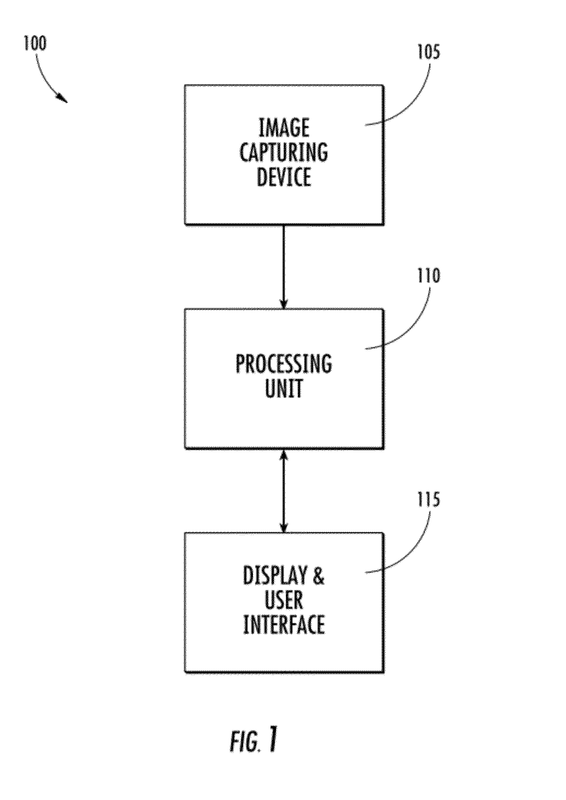

Referring now in more detail to the drawings, the invention will now be further described. As shown in FIG. 1, a system and method for the non-contact measurement surfaces are disclosed. The measurement system 100 comprising an image capturing device, which can capture images, for example a target object on a target surface. For example, the target surface can be a wound on a biological surface, such skin. The target surface, in another example, can be a defect in a non-biological surface, for example and not limitation, a dent in a car bumper. According to some embodiments of the invention, a system for the non-contact measurement of lesions and wounds are disclosed. In an preferred embodiment of the present invention, the wound measurement system 100 of the present invention comprises an image capturing device 105, which can capture images, for example images of a wound. The image is then sent to a processing unit 110. The software of the processing unit utilizes a computer vision component, provides the user with a suggested boundary for the wound, and calculates the real world area of the wound based on this boundary. These calculations are transmitted to the display and the user interface 115. The display and user interface 115 allows the user to accept, reject, or modify the given boundary provided by the processing unit. As the user modifies the wound boundary, the processing unit continues to provide calculations of the area enclosed.

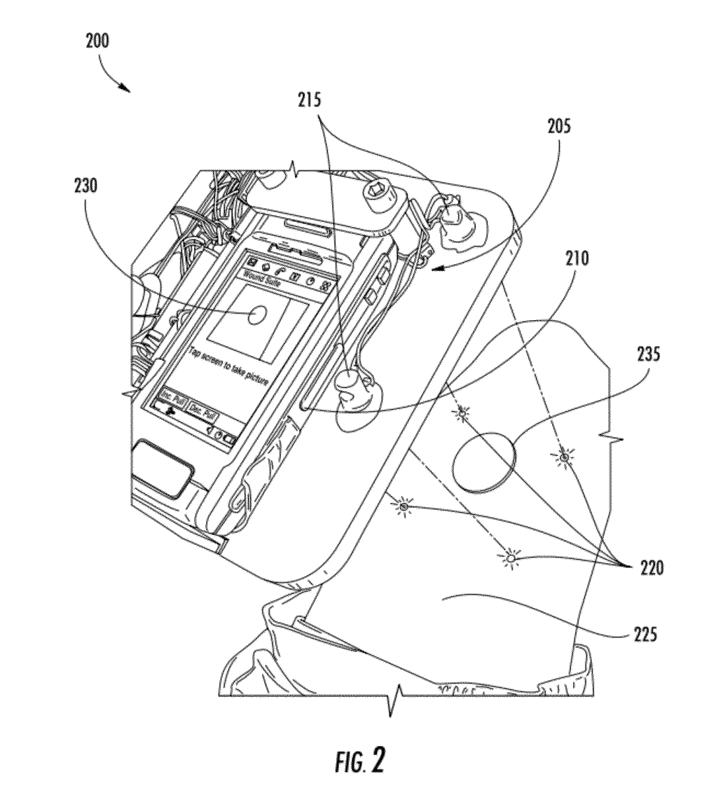

The present invention for wound measurement is further described in FIG. 2. A wound measurement system 200 utilizes an image capturing device 205 comprising an image capturing element 210 and a laser element 215. Preferably, there are at least four laser elements 215; however, two sets of four laser elements can be used to further accommodate the variable size of wounds. In such an embodiment, each of the four laser elements 215 can be positioned equidistantly from the image capturing element 210 so that each of the four laser elements 215 comprise a corner of a square surrounding the image capturing element 210. Each of the lasers elements 215 individually projects a light, preferably in the shape of a dot, 220 on the target surface 225. Initially, the image capturing device 205 can present the user with a viewfinder/user interface 230 showing what the image capturing element 210 sees. The user identifies the wound 235 and then captures an image of the wound where the wound 235 occupies as much of the image as possible and the laser-created dots 220 are still within view on the viewfinder/user interface 230. The image capturing device 205 further comprises a processing unit. The processing unit of the image capturing device 205 can comprise a computer vision component. The viewfinder/user interface 230 is preferably a touch screen, permitting user modification of the detected wound boundary.

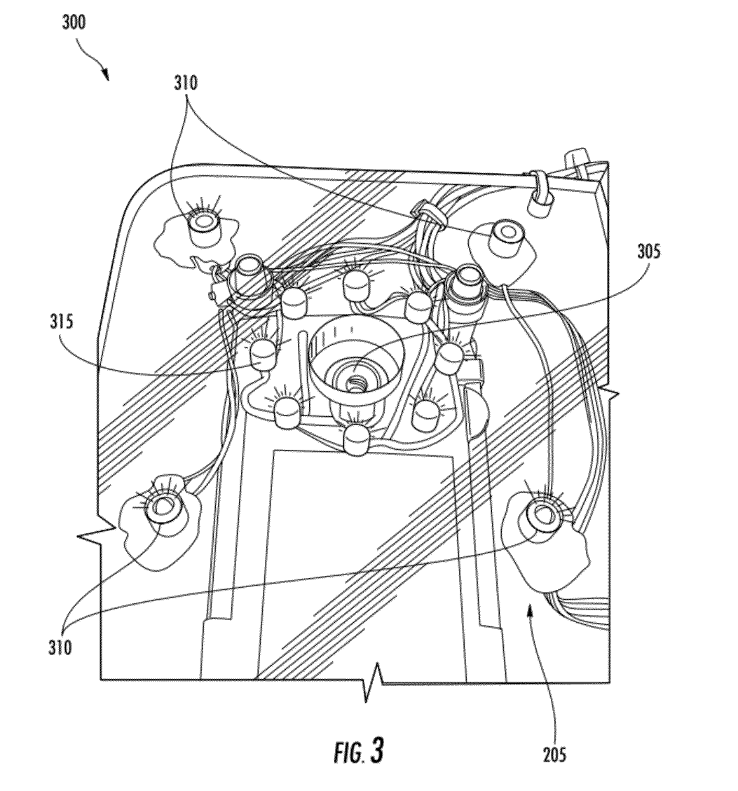

FIG. 3 further illustrates the image capturing device 300. In the present embodiment, the image capturing device 205 comprises an image capturing element 305, a plurality of laser elements 310, and an auxiliary lighting element 315. Preferably, there are at least four laser elements 310. The four laser elements 310 can be positioned parallel to one another at known locations around the image capturing element 305. In the present embodiment, each of the four laser elements 310 is positioned equidistantly from the image capturing element so that each of the four laser elements comprise a corner of a square surrounding the image capturing element 305. The fixed location of the laser elements 310 relative to the image capturing element 305 permits for computation of range finding and skew calculations. An auxiliary lighting element 315 can be located adjacent to and arrayed around the image capturing element 305 so as to illuminate the target surface. The use of auxiliary lighting allows for capturing wound images in both well-lit and dark ambient conditions. Further, the addition of an additional laser line element (not pictured in the present embodiment) permits calculation of wound depth.

In an exemplary embodiment, a Sony Ericsson P900 camera phone can function as the image capturing element. Many digital cameras, including those found in cell phones and personal digital assistants (PDAs), can serve as the image capturing element. The image capturing device can perform image capture, image processing through the use of computer vision techniques, and most user interactions. In an exemplary embodiment, a dedicated microprocessor-based system with a camera and touch screen can function as the image capturing device. In another embodiment, a mobile computing platform can function as the image capturing device. The data collected by the image capturing device can be transmitted or transferred to additional data analysis devices by both wired and wireless networks including for example and not limitation Bluetooth, IEEE Standard 802.11b, or through data storage devices, such as memory storage cards.

Software on the Sony Ericsson P900 camera phone can be written in C++ and makes use of Symbian and UIQ infrastructure to access the camera and provide a user interface. When the user initiates image capture, the phone captures a 640×480 RGB color image. In one embodiment, the image can be then scaled down to 320×240 to provide enough information for the computer vision component while significantly decreasing the processing time when Bluetooth communication is utilized. In the preferred embodiment, there is no need to scale the image as the image capturing device and processing unit comprise a single self-contained device. Further, there is no need to scale the image when the image is transferred wirelessly to a server, computer or a memory storage device. Before the image is transferred to the processing unit, the image capturing device attempts to find the four laser points. If the laser points show that the image is too skewed to provide an accurate area estimate, the interface can prompt the user to take another image. In some cases, depending on wound location, this may not be possible and the user is given the choice to override this decision. The captured image is then transmitted to the processing unit.

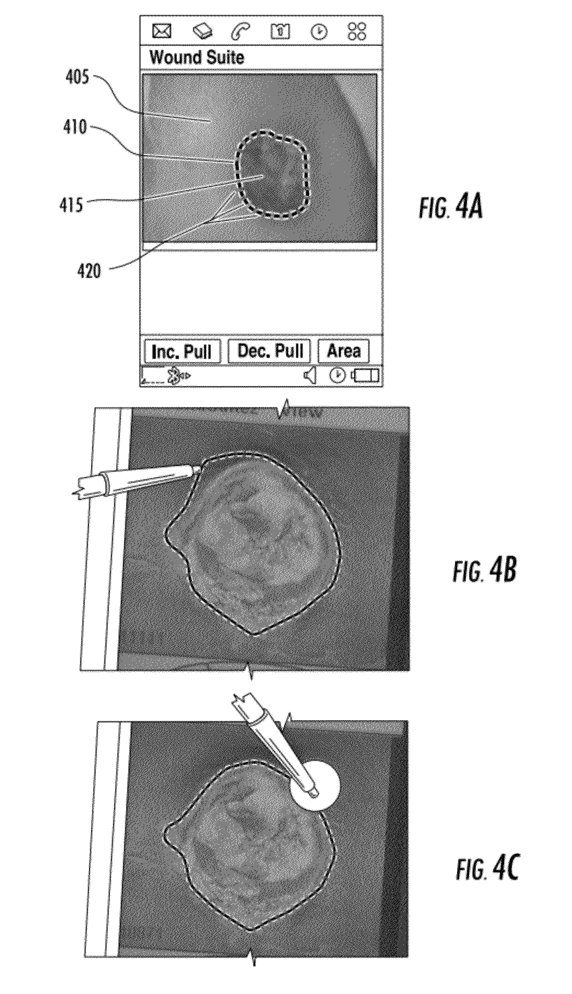

After capturing the image of the wound with the image capturing device 305, the image is transferred to the processing unit and analyzed by a computer vision component. The computer vision component returns a boundary of the wound to the user interface along with information relating image dimensions to real-world measurements. FIG. 4A demonstrates a screen capture 405 of a detected wound boundary 410 with the computer vision component. The results of the analysis by the computer vision component are displayed to the user in the form of a boundary 410 drawn on top of the original image 415. The boundary comprises a number of control points 420. The boundary of the wound can be modified by the user. If the user selects a single control point, the predicted boundary of the wound can be “dragged” as illustrated in FIG. 4B. Alternatively, if the user selects an area outside the boundary of the wound, the position of several control points can be concomitantly modified and the predicted boundary of the wound can be “nudged” as illustrated in FIG. 4C. In the present embodiment of the invention, the number of control points that can be concomitantly modified by “nudging” can be modified thus providing a tunable control for predicted boundary modification. In addition to being able to modify the predicted wound boundary, the user can redraw the wound boundary by hand through the use of a stylus in the instance when the computer vision component cannot isolate a wound boundary. The interfacing code can be written using C++ or C# (C-sharp).

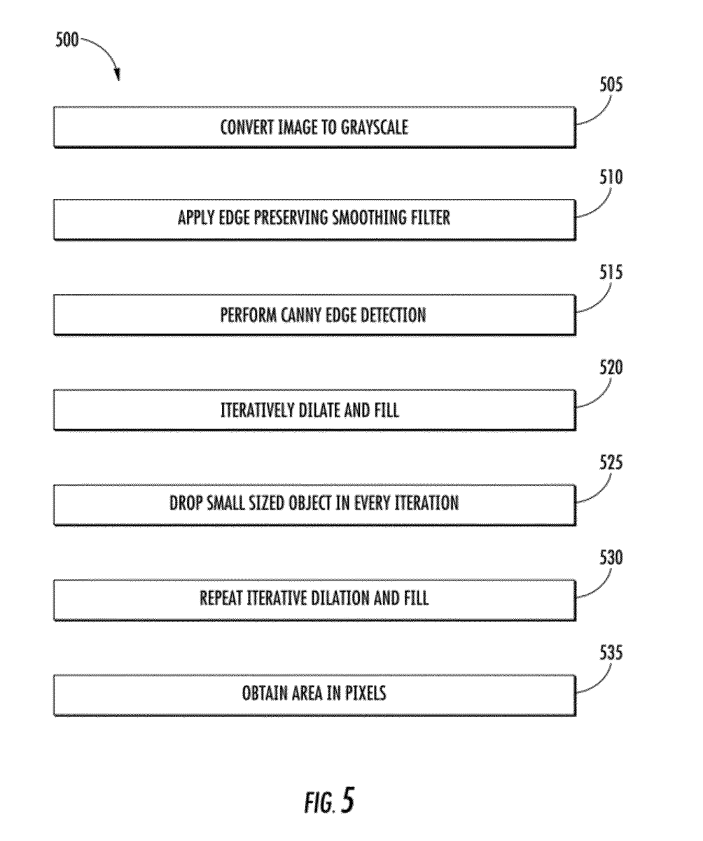

The computer vision component of the processing unit employs the boundary detection algorithm illustrated in FIG. 5. At 500, the boundary detection algorithm can use an edge detection based segmentation method to identify the boundary of the wound. At 505, the captured image is converted into a grayscale image by creating a weighted combination of the red, green, and blue color channels. Then at 510, an anisotropic smoothing filter can be applied to smooth image regions while preserving edges, so as to get better results in the edge detection stage. Next at 515, the Canny edge detector can be applied to the image to identify boundaries. Then at 520, the connected wound boundary can be obtained by iteratively dilating and filling the edge map. At 525, objects with size below a certain threshold in the image are dropped at every iteration. As shown in 530, the process of iteratively dilating and filling the edge map and dropping small sized objects at every iteration is continued until a large connected region is obtained. Then this connected region can be eroded and smoothed to create the final segmentation. At 535, the area obtained at this stage is the area in pixels 535.

To correlate the area in pixels of the captured image to the real area of a wound, an image of known dimensions is projected on or near the wound using laser pointers. The known projection can then be captured along with the wound by the image capturing element. The known projection is then identified in the captured image. Using the size of the projection, the correlation between pixel area and actual area can be obtained. Apparent distortion in the image from the known shape can be used to compensate for cases where the camera has not been held exactly parallel to the wound surface through image registering.

Preferably, the image of known dimension is a laser-created dot. Four parallel laser pointers can project four dots on to the skin to form the boundaries of a square-shaped image. The laser dots in the image are identified using a two-step approach. First, thresholding is used to identify potential laser dots based upon intensity. Then, a probabilistic model is used to select the four most likely points based upon shape, size and location inputs. The relative positions and the distance of the dots from each other can be used to find the distance and orientation to the wound, to calculate the area of the wound and to correct for any positioning inaccuracy.

The computer vision component of the processing unit can be written in C# or MATLAB and can have at least two stages: (1) unskewed image to establish a mapping between physical size and the imaged size, and (2) detect the wound boundary.

The image is first unskewed using the four laser dots. For detecting the laser dots in the image, the laser dots are identified using a two-step approach: (1) thresholding is used to identify potential laser dots based upon intensity, and then (2) a probabilistic model is used to select the four most likely points based upon shape, size and location inputs. Each of these four points is taken as the coordinates of a laser dot.

If the skew is greater than a particular threshold, then the skew correction procedure outlined below can be used. Otherwise, the pixel distance between the detected laser points is found, and this distance is directly correlated to the known distance between the projected laser points in the image. To detect whether the skew is too high, a simple scheme is defined. A quadrilateral is defined by the laser points found in the image. The deviation from the mean length is calculated for each side. If this deviation is greater than a threshold then the skew correction procedure is used. While this technique might not be an exact measure of the skew, it gives a good enough estimate for whether to eliminate the skew correction step.

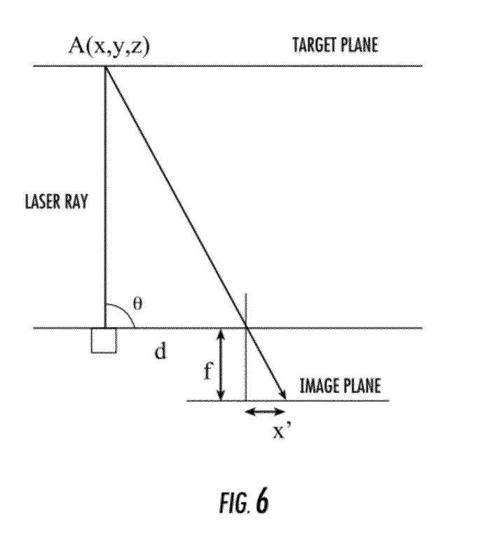

To correct for the problem of the image capturing element not being parallel to the target plane, the correspondence between the target plane being imaged and the image taken by the camera must be determined as illustrated by FIG. 6. Using the fact that the laser pointers and camera have a fixed, known orientation with respect to each other, the real-world coordinates of the laser points can be calculated. Then, the distance to the wound plane can be determined using triangulation. Using simple geometrical relations, one can establish the below formula (hereinafter Formula 1)

$$[ x , y , z ] = \frac{d}{f cot θ - {x}^{1}} [ {x}^{1} , {y}^{1} , f ]$$

where d is the X axis distance from the camera center to x, θ is the angle made by the laser ray to the camera plane, f is the focal length of the camera, A(x; y; z) is the true world coordinates of the point in the camera coordinate system, and x′ is the X-axis measure of the imaged point.

For calibration of the system, the intrinsic calibration parameters are determined using the method given by Zhang et al., Border Detection on Digitized Skin Tumor Images in IEEE Transactions on Medical Imaging, 1128-43, 2000. This method provides five distortion parameters k₁-k₅, focal length (f) of the camera, and the camera center coordinates, which may be different from the center pixel on the image. The laser pointers are only approximately orthogonal to the image plane so the parameter θ needs to be evaluated. To obtain the parameters d and f cot (θ), images at known heights are taken and the system is solved for df and f cot (θ). From the camera calibration, f is known, and hence d can be obtained. Both these calibrations have to be done only once for a given system.

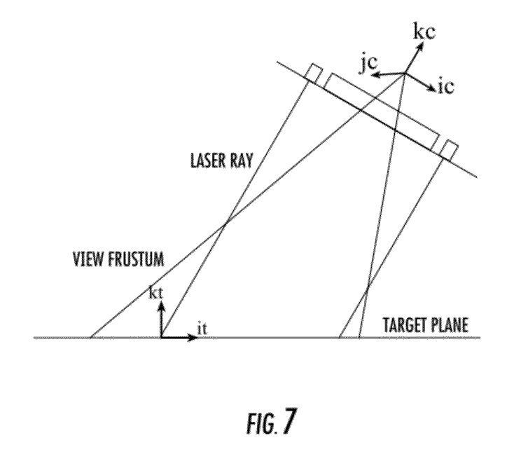

To correct the skew, first the coordinates of the laser dots are found in the camera's coordinate system using Formula 1. To get a more accurate measure, a similar calculation can be done using y instead of x and the average of both is calculated. A 3D coordinate system is established such that the X and Y axes of the system lie in the target plane. This coordinate system will be referred to as the target coordinate system. To determine the laser positions in the target coordinate system as illustrated in FIG. 7, a rotational matrix and translational offset is established between the two systems and the vectors for the laser positions are transformed into the target coordinate system using the below formula (hereinafter referred to as Formula 2).

$$Xt = ( \begin{matrix}R & t \\ 0 & 1\end{matrix} ) ( \begin{matrix}Xc \\ 1\end{matrix} )$$

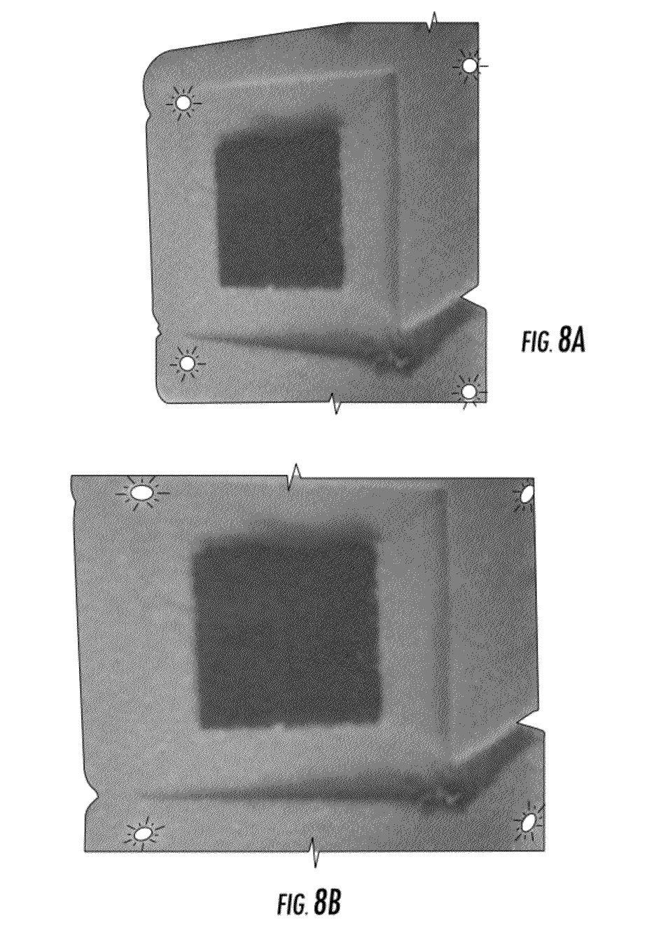

Xc and Xt are camera and target system coordinates of point X, R and t are the rotational matrix and the translation matrix, respectively. R is constructed by using the projections of it, jt, kt in the camera coordinate system as rows. t is the origin of the camera coordinate system expressed in the new target coordinate system. The positions of the laser points are now mapped onto a discrete image grid. Using the four laser points position vectors in this image grid and in the image captured by the camera, we can use a projective transform to map the rest of the image onto the target image grid. FIG. 8A illustrates an original skewed image, whereas FIG. 8B illustrates an unskewed image using the above calculations.

The next step is to segment the wound out of the image. For segmenting a pressure ulcer, Jones and Plassmann suggest an active contour model. See Jones & Plassmann, An Active Contour Model for Measuring the Area of Leg Ulcers in IEEE Transactions On Medical Imaging, 1202-10, 2000. This model was observed to have some practical limitations. The wound boundary detected varied with the initial (or seed) boundary approximation selected. Varying factors, such as wound size and shape along with the distance of the camera to the wound plane, make it difficult to choose a single initial boundary. Additionally, the wounds generally have many edges which are not a part of the boundary causing the active contour to stick to these “false edges.” Zhang et al. alternatively proposed a radial search method for detecting skin tumor images.

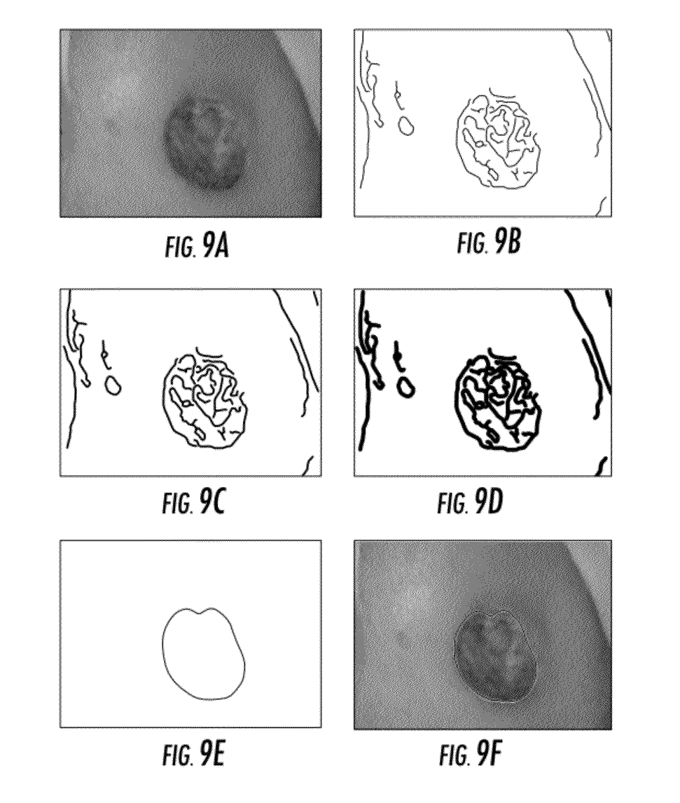

The present invention can utilize an edge detection based segmentation algorithm. The boundary detection algorithm implemented in the present invention uses an edge based segmentation method to identify the boundary of the wound. FIGS. 9A-9F illustrate the processing progressions as the algorithm is applied to locate a large connected object in the image. First, the captured image is converted into a grayscale image by creating a weighted sum of the red green and blue channels as illustrated in FIG. 9A. An anisotropic diffusion smoothing filter which preserves edges is then applied to smooth noisy image regions while maintaining edges. This reduces false edges in the edge detection stage. A Canny edge detector is then applied to the image to identify potential wound boundaries. At this stage the resulting edge map will still have many false edges and breaks in the image boundary as illustrated in FIG. 9B. The binary edge image is then dilated after which the algorithm fills in all background pixels completely surrounded by a boundary. This process will fill the wound when a connected wound boundary is returned by the dilation. The dilation and filling of the edge map is continued iteratively until a large enough connected boundary is obtained. In every iteration, small sized objects in the image are dropped. When a large enough connected region is obtained, the binary image is eroded to correct for increased size during dilation, and then smoothed using a median filter. FIGS. 9C-9E illustrate a filled image after 2 iterations, an edge map after 3 iterations, and a segmented image after four iterations, respectively. The final area obtained at this stage is the wound area in pixels. FIG. 9F demonstrates a segmented boundary superimposed on the original image.

These and other objects, features, and advantages of the present invention will become more apparent upon reading the following examples.

### EXAMPLE 1

Not all wounds, however, will be easily found by the computer vision component. In this case, the judgment of the wound boundary is left up to the user of the device. The user can be prompted to draw a boundary around the wound. As previously stated, repeatability of measurements is more important than absolute accuracy when monitoring wound progress. While the same user may be able to make the same measurements repeatedly with existing methods, it is quite difficult to ensure that multiple users will take measurements in the same way. For example, in the ruler based methods, it is quite common for different users to choose different directions for the maximum diameter of the wound.

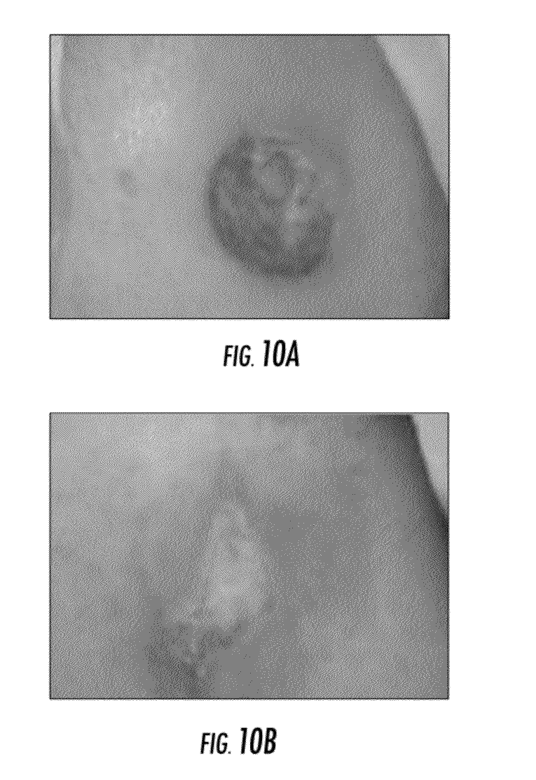

In order to develop a better understanding of the issues with repeatability when tracing the wound in our interface, we performed an experiment involving three members of the design team and two wound images as illustrated in FIG. 10A and FIG. 10B. First, each user was given a demonstration of how to use the application. Then, each user was asked to trace each wound image ten times. The user alternated between wound images, tracing one and then the other. Modification of the boundary was possible by pulling the control points. The user was asked to signal when they felt they had accurately surrounded the wound. The user was never allowed to see the actual area enclosed to prevent them from trying to match it every time. Because of screen size limitations the image displayed was sized down to 200×150. The boundary created by the user is then scaled up to the corresponding points on the 320×240 image to determine how many pixels were enclosed in the image used for actual area measurements. This leads to the user having to enclose pixels in a lower resolution space than the one used to calculate the area. Table 1 presents the mean and coefficient of variation for the number of pixels bounded by each user per wound image.

TABLE 1

| User | Image 1 | Image 2 |
|---|---|---|
| 1 | 9603.0 (2.13%) | 5839.5 (8.68%) |
| 2 | 10380.4 (4.53%) | 8439.6 (9.99%) |
| 3 | 10458.0 (6.84%) | 7596.2 (7.71%) |

The data presented in Table 1 demonstrates that even novice users were capable of repeatedly tracing the wound with high accuracy. The inter-rater differences are attributable to the fact that the novices are not professional wound care specialists and therefore have very different ideas of what exactly constituted part of the wound. In addition, the second image was purposefully chosen because of the difficulty associated with determining its boundary.

### EXAMPLE 2

To test the computer vision component, two tests were performed. A square (3.8 cm×3.8 cm×0.1 cm) was cut into green foam. The surface of the square was painted brown. To test how the algorithms respond to changes in the camera-to-wound distance, the wound detection unit was mounted on rig with a vertically movable platform. Using the movable platform, the foam wound shape was photographed from various heights and the computer reported area was recorded for both the simple distance correlation and skew correction schemes. The results are shown in Table 2.

TABLE 2

| | Area using Skew | Area using Direct |
|---|---|---|
| Distance (cm) | Correction (cm²) | Correlation (cm²) |
| 17 | 13.54 | 14.25 |
| 20 | 13.35 | 14.25 |
| 25 | 13.56 | 13.72 |
| 30 | 13.64 | 13.40 |

The mean of the area by triangulation approach is 13.76 cm² with a standard deviation of 0.485 (3.52% as a percentage of the mean). This indicates a high value of repeatability. The difference of the mean compared with actual known area to known area is about 6.3%. For the direct distance correlation method the mean is 13.86 cm² with a standard deviation of 0.3375. The area measurements in the direct distance calculation have an average error of 3.7%.

### EXAMPLE 3

For quantifying the effect due to skew, the device was mounted on a bar that could be rotated through various angles along a single axis which was orthogonal to the camera's line of sight. The foam wound was photographed for 2 different heights and from various angles. Table 3 gives the area values reported.

TABLE 3

| Angle° | Dist = 19.5 cm | Dist = 17.7 cm |
|---|---|---|
| 0 | 13.64 | 13.71 |
| 10 | 13.17 | 13.85 |
| 15 | 13.22 | 13.81 |
| 20 | 13.86 | 14.31 |
| 30 | 14.08 | 14.62 |
| 35 | 13.31 | 14.51 |

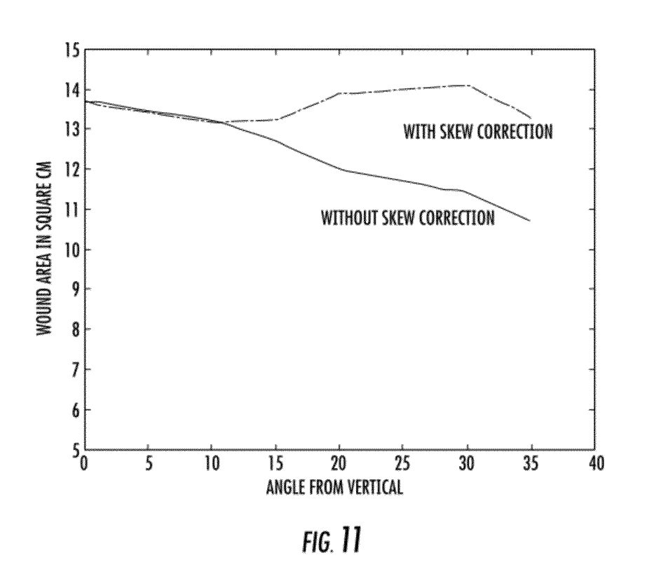

The mean is 13.84 cm² with a standard deviation of 0.457 (3.3% as a percentage of the mean). Comparing these values to values from Example 2, the standard deviation value of 0.420 obtained from the present experiment is similar to the one obtained when the camera was kept exactly horizontal. Thus, almost the whole error due to the skew was corrected for in the range of angles 0° from vertical to 35° from vertical. FIG. 11 illustrates the area measurements as skew increases. FIG. 11 further demonstrates the difference between when the skew correction procedure is used and when it is not used. The two lines in FIG. 11 show the determined area as a function of angle for the height 19.5 cm. It is observed that the mean calculated for the case when skew correction is not used is 12.31 cm² and the standard deviation is 1.1019 (9% as percentage of mean). The maximum difference of a reading from the exactly orthogonal case for skew corrected readings is 0.47 while for non skew corrected it is 3.05.

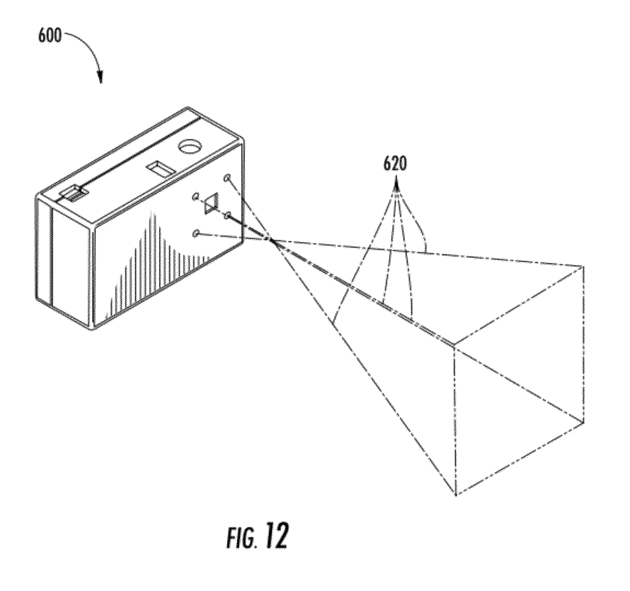

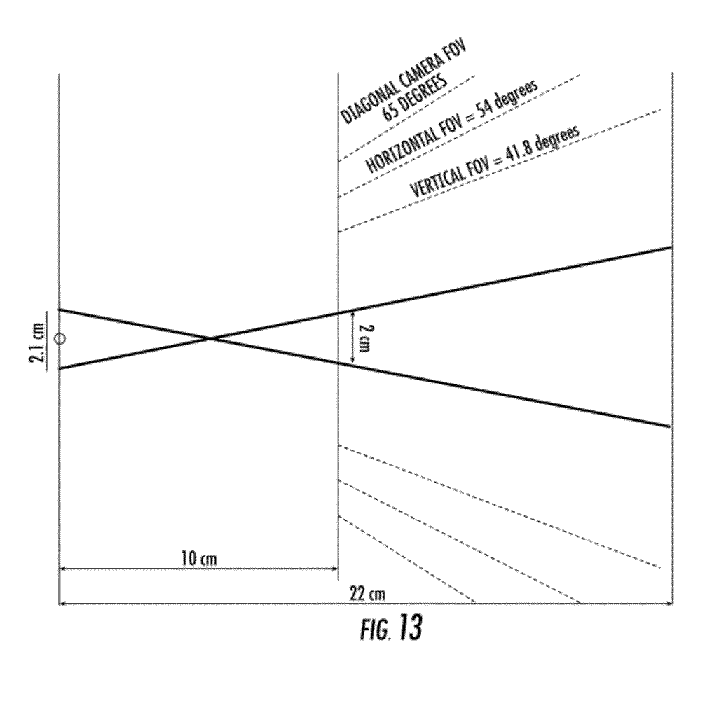

As illustrated in FIGS. 12-16, an alternative embodiment of the wound measurement system 600 uses a divergent configuration of laser elements 620 (see FIG. 12), which accommodates a much wider range of target object sizes than a set of parallel laser elements. Inside the working volume of the device, target objects can be measured ranging in size from smaller than 2 cm square (see FIG. 13) to an area greater than 10 cm square. The flexibility in target object size afforded by the divergent laser configuration (e.g., schematically illustrated in bold solid line in FIG. 13) may greatly increase the effective application of the invention.

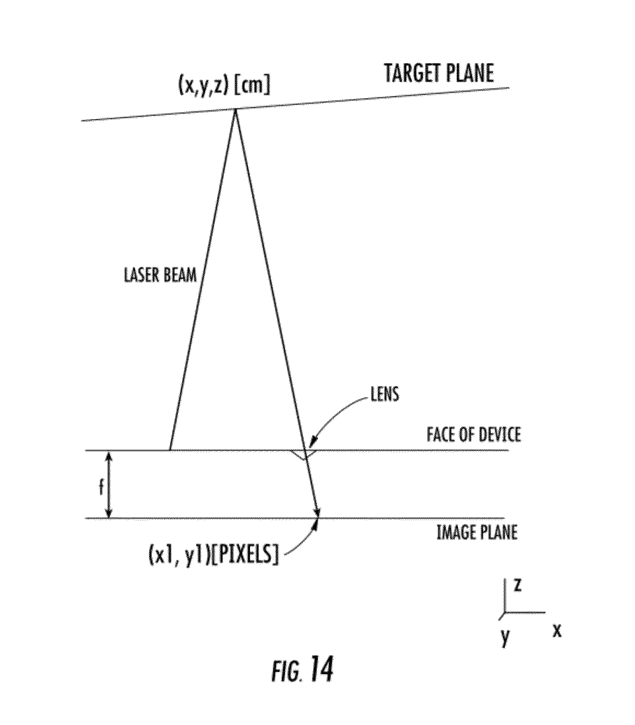

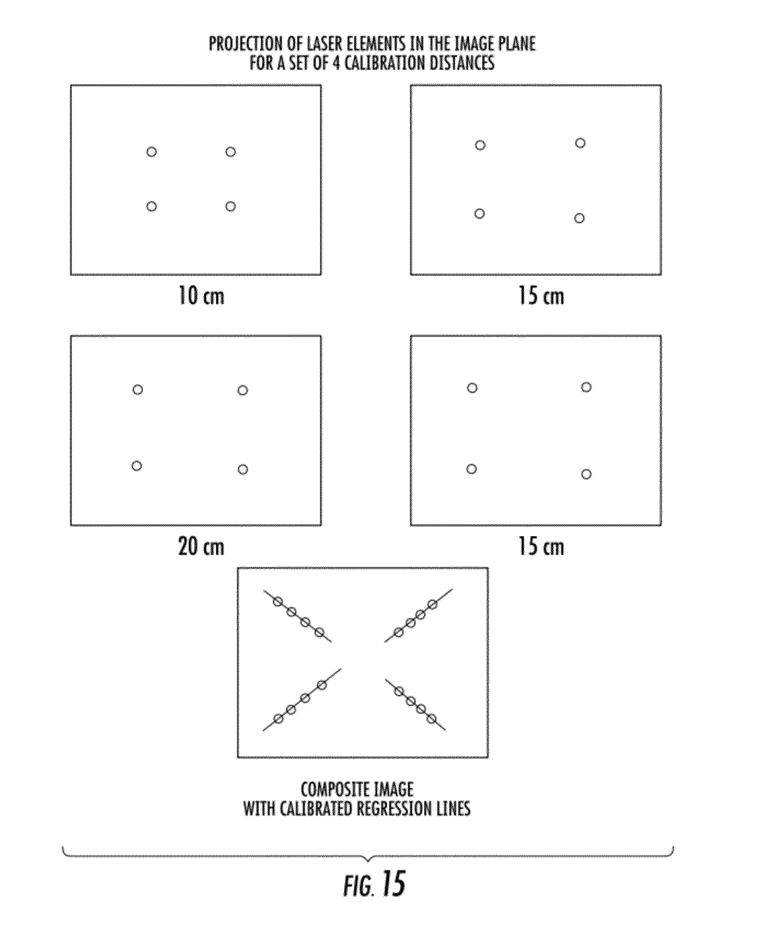

If the laser elements diverge at an angle different than the field of view of the camera, then there is a unique mapping of laser elements from image coordinates (x1, y1) (e.g., in pixels) to real world coordinates (x, y, z) (e.g., in cm) (see FIG. 14). The angle of the laser elements is constrained by the field of view of the camera, and by the range of sizes of the target objects. Note that the lasers may be inclined either towards or away from the axis of the image capture device. The illustrated embodiment inclines the lasers at an angle of 18 degrees towards the axis of the image capture device but to accommodate target objects of varying size within the working volume of the device, the inclination angle of the lasers may range anywhere from 30 degrees away from the axis of the camera to 60 degrees towards the axis of the camera. FIG. 15 illustrates the unique mapping of laser elements onto the image plane for a set of four calibration images when using a divergent set of laser elements.

Real world coordinates (x, y, z) are associated with image pixel coordinates (x1, y1) and can be determined using a formula in the following form (hereinafter referred to as Formula 3):

$$[ x , y , z ] = \frac{A}{( {x}^{1} + B )} [ {x}^{1} , {y}^{1} , f ]$$

The two calibration parameters A and B are independent of the optics of the camera and can be determined from a set of four calibration images using linear regression. There are unique values for A and B for each laser element, and for each pixel coordinate (x and y).

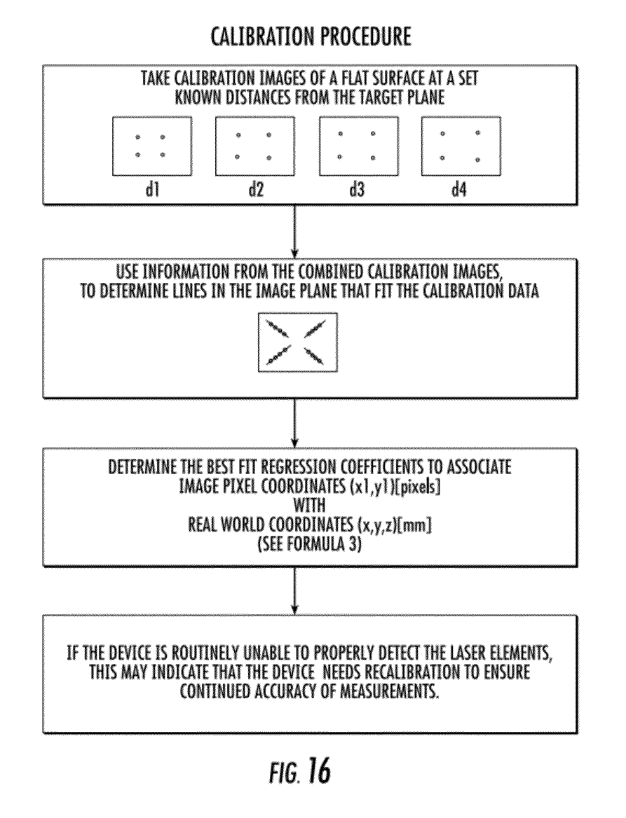

The calibration method of the current embodiment may be accomplished by taking a number of images be taken of flat surfaces from a set of known calibration distances (see FIGS. 15-16). As shown in FIG. 15, there is a unique relationship between pixel coordinates of the laser elements and the known real world coordinates of the calibration laser elements. Using the pixel coordinates of the laser elements in the calibration images, the known real world coordinates of the laser elements with respect to the device, and the form of the trigonometric transformation (using Formula 3), the coefficients A and B can be determined with a simple linear regression model. Because these parameters are determined with regression, properties of the device (such as principal point of the image capture device, and the precise angle of inclination of each laser element with respect to the plane of the image) are hidden, and lower error margins are achieved.

This optics-agnostic approach to calibration decreases the error propagation in the calculation of surface properties by reducing the total number of measured parameters, and by relying on regression instead of direct measurement. This calibration technique also allows for a variety of laser configurations without modification to Formula 3 (e.g., sign changes that may result from a crossing laser pattern).

As seen in FIG. 13, the laser elements are located in a tightly constrained region of the image plane, and each falls along a straight line. Each laser element is calibrated using its own regression line, and the transformation to real world coordinates is independent of all other laser elements. A minimum of three laser elements are required to identify a target plane in the real world. In the illustrated embodiment, four laser elements are used to increase accuracy in identification of a target plane.

Using information from the device calibration to reduce the area of the image that is searched for laser elements, it is possible to automatically identify the laser centroids (i.e., center of each laser element), as projected on the 2D image plane, with a high degree of accuracy. This enhances the usability of the device as it is less likely users will be required to correct for a falsely identified laser element.

Furthermore, if the user must correct the location of the laser elements identified, it may alert the program that the device is in need of calibration. This ensures continued accuracy and provides an automatic check of the device calibration which will become important with continued use.

### EXAMPLE 4

The divergent lasers accommodate a wide range of target object sizes on surfaces of varying curvature. In the case of wound measurement, for example, large wounds on a relatively flat surface (e.g., the back) would require a relatively large configuration of laser elements. With a diverging laser configuration, this can be achieved by moving the device farther away from the target plane. However, small wounds located on a surface of high curvature, such as the back of the heel, require the laser elements be tightly clustered around the wound. This can be achieved with the current embodiment by moving the device closer to the target plane. This flexibility reduces practical constraints on the use of the device in the field.

Use of a high-resolution 5MP camera allows for much more accurate identification of the wound boundary. This allows the user to zoom in during border determination to decrease uncertainty in wound measurements. The increased image resolution also allows for more accurate calculation of real world coordinates and identification of laser elements in the image.

It should be appreciated that the user can interact with the displayed image to circumscribe the wound border or modify the border as defined by the processing unit, like in the first embodiment described above. Moreover, it should be understood that it is possible that a combination of parallel and divergent lasers may be utilized (i.e., all lasers need not intersect).

In accordance with the provisions of the patent statutes, the principle and mode of operation of this invention have been explained and illustrated in its preferred embodiment. However, it must be understood that this invention may be practiced otherwise than as specifically explained and illustrated without departing from its spirit or scope.

What is claimed is:

1. A portable, hand-held, non-contact self-contained surface measuring system capable of providing quantitative measurements of a target object on a target surface, the system comprising:
an image capturing element for capturing an image of at least a portion of the target object;
at least four projectable reference elements for defining at least one characteristic of at least a portion the target object, the projectable reference elements comprising a divergent configuration of lasers;
a processing unit; and
a user interface for displaying the captured image.
